(19) United States
(12) Patent Application Publication
(10) Pub. No.: US 2016/0130546 A1
(43) Pub. Date: May 12, 2016

(54) PHOTOBIOREACTOR SYSTEM AND METHOD

(71) Applicant: NEXGEN ALGAE, LLC, Columbia, MD (US)

(72) Inventor: Wendell B. Leimbach, Saint Leonard, MD (US)

(21) Appl. No.: 14/904,230

(22) PCT Filed: Jul. 10, 2014
(86) PCT No.: PCT/US2014/046196

§ 371 Date: Jan. 11, 2016

Related U.S. Application Data
(60) Provisional application No. 61/845,786, filed on Jul. 12, 2013.

(51) Int. Cl. C12M 1/02; C12M 3/00; C12M 1/34; C12M 1/00; C12M 1/42

(52) U.S. Cl. CPC C12M 27/00; C12M 21/02; C12M 29/00; C12M 35/02; C12M 23/22; C12M 41/06; C12M 41/42; C12M 23/38; C12M 23/46; C12M 23/58

(57) ABSTRACT

Embodiments include a photobioreactor system for incubating algae growth medium. The photobioreactor system can include a plurality of photobioreactors. Each photobioreactor can include an enclosure for receiving, incubating, agitating, and with-drawing algae growth medium. A plurality of aeration apertures positioned within each enclosure can deliver air bubbles to agitate the algae growth medium. An aeration apparatus can be connected to the plurality of aeration apertures in each photobioreactor. The photobioreactors can be positioned with respect to each other to permit the gravity flow of algae growth medium from the outlet port of each photobioreactor. One or more light sources operatively and controllably associated with the photobioreactors can emit electromagnetic radiation directed toward the enclosure. Embodiments also include methods of receiving, incubating, agitating, and withdrawing algae growth medium.

### RELATED APPLICATION

This application claims priority to provisional application No. 61/845,786, filed on Jul. 12, 2013, the disclosure of which is hereby incorporated by reference in its entirety.

### FIELD

The present disclosure generally relates to systems and methods for growing algae to produce algal biomass.

### BACKGROUND

Micro-algae are one of the fastest growing species of algae. They grow and multiply rapidly, while photosynthesizing carbon dioxide, water, and sunlight into biomass and oxygen. Growth of micro-algae can be useful for producing biomolecules such as such as Astaxanthin from Haematococcus pluvialis (Hp), long chain (LC) omega-3 fatty acids from Nannochloropsis limnetica, and beta glucan from Euglena gracilis. Algal biomass is typically used as a food or dietary supplement, as fertilizer or livestock feed, and as a feedstock for biofuels. In many cases, an algae production facility can produce algal biomass directed to many different uses, as byproducts of the same system. For instance, algal biomass can generate biofuels such as biodiesel. Algae can also preserve marine ecosystems by recycling waste from such ecosystems (e.g., fish waste) and converting waste into feedstock for fish. Algae also recycle carbon dioxide into oxygen, thereby mitigating levels of anthropogenic carbon dioxide emissions.

Algae can grow in a variety of sources (e.g., fresh, brackish, marine and wastewater) and in enclosed systems known as photobioreactors. One example of an micro-algae species that can be grown in an open system (e.g., a pond or tub) is Spirulina. Such open systems are simple to operate and have a lower capital and maintenance cost. However, certain species of micro-algae (e.g., Hp) have a more fragile cell wall structure, thereby requiring a more carefully controlled growth medium. Such species are typically grown in closed systems. One example of a closed system is a photobioreactor system. Photobioreactor systems are capable of minimizing water and land use.

### SUMMARY OF THE INVENTION

Certain embodiments of the invention include a photobioreactor system for incubating algae growth medium. The photobioreactor system can receive, incubate and agitate an algae growth medium (e.g., water injected with algae and optionally any nutrients). The photobioreactor system can include a single photobioreator, but preferably includes a plurality of photobioreactors. Each photobioreactor has an enclosure for to receiving, incubating, agitating, and withdrawing algae growth medium. A plurality of aeration apertures can be positioned within each enclosure to deliver gas (e.g., filtered ambient air) bubbles in a manner sufficient to agitate the algae growth medium. The photobioreactors, in turn, are preferably supported and positionable (in or able to be positioned) with respect to each other in a manner sufficient to permit the gravity flow of algae growth medium from the outlet port of a first photobioreactor to the inlet port of a second photobioreactor. One or more light sources can be operatively and controllably associated with the photobioreactors to emit electromagnetic radiation directed toward the enclosure.

Micro-algae can be grown in photobioreactors in which a medium for growing algae can be exposed to light, nutrients, and carbon dioxide. Conventional photobioreactors typically include an enclosed volume of a growth medium, into which algae can be introduced. Algae tend to grow on surfaces of such photobioreactor, thereby preventing algae that are far away from the surface of the photobioreactor (e.g., in the interior of an enclosure) from access to light. Several algae species also have a tendency to self-flocculate and settle at the bottom of a photobioreactor system. In such cases, algae can grow and block various ports for circulating the growth medium (e.g., water, air, and/or nutrient ports). Such conditions can result in poor algal density and limited exposure to light. Also, typical polymers used for constructing photobioreactors can leach into the growth medium, thereby contaminating it. Such conditions can suppress algae growth. Applicant has discovered the manner in which these and other concerns arise, and in turn, can be addressed in the manner described herein.

In some embodiments, the photobioreactor system can include an aeration apparatus operatively and controllably connected to the plurality of aeration apertures in each photobioreactor.

In some embodiments, the plurality of aeration apertures are, independently, each of a diameter in the range of about 0.005 centimeters to about 0.1 centimeters, and preferably about 0.01 centimeters to about 0.05 centimeters, e.g., when used for an algal density of about 0.5% by dry weight. In turn, the end-to-end spacing between adjacent aeration apertures is independently, and preferably, between about 0.01 centimeters to about 1 centimeter, and more preferably between about 0.05 centimeters to about 0.1 centimeter. For instance, a standard photobioreactor of the type described herein (e.g., on the order of 0.5 meters to about 10 meters in overall length, or more preferably on the order of about 1 meter to about 2 meters in length) can have between about 100 and about 1000, more preferably between about 200 and about 800, and even more preferably between about 300 and about 600 aeration apertures per photobioreactor. The apertures can be of any suitable combination of numbers, diameters, spacing and orientation.

In some embodiments the plurality of aeration apertures is controllably operated in a manner sufficient to agitate the algae growth medium, and in turn, to minimize clogging of the aeration apertures by the algae growth medium. Preferably, the plurality of aeration apertures can deliver air bubbles in a turbulent manner, providing sufficient turbulence to minimize or substantially prevent algae from settling at the bottom of the tank, while at the same time substantially preserving the cell wall structure of various types of algae contemplated. Depending on various parameters (e.g., aperture size, gas flow, and the geometry within the enclosure), the bubbles can take on the order of seconds to circulate (e.g., move from the interior toward the exterior) within the photobioreactor. For instance, the bubbles can have a residence time within the enclosure of between about ½ and about 5 seconds, and more preferably between about 1 and about 2 seconds.

Certain embodiments of the photobioreactor can include a base panel and a cover. The base panel and the cover each can have an edge perimeter and an end perimeter. The base panel and the cover can be sealably connected to each other at their respective edge perimeters to form a fluid tight seal, such that they define the substantially fluid tight enclosure. In certain embodiments, the base panel of the photobioreactor is sloped such that it forms an angle in the range of about 1 degree to about 20 degrees, and more preferably between about 2 degrees to about 10 degrees, with a substantially horizontal plane, such as the floor of a supporting structure. In some embodiments, the aeration apparatus generates bubbles moving in an upwardly direction from the base panel and toward a ridge of the cover. The aeration apertures can be defined in a channel recessed in the base panel. In some embodiments, the base panel and the cover are sealably connected by the use of appropriate means, e.g., silicone adhesives, and/or a gasket.

Certain embodiments of the photobioreactor system can include one or more water flow pumps in fluid communication with each photobioreactor. A water flow pump suitable for use in the present invention can move the growth medium from the downstream outlet of a photobioreactor to an upstream photobioreactor, or from the outlet port of a photobioreactor to an upstream entry port of the same photobioreactor. In turn, in a preferred embodiment, connecting tubes can be coupled to a fluid line for maintaining suitable (e.g., atmospheric) pressure in each of the plurality of photobioreactors.

In some embodiments, each of a plurality of photobioreactors has a triangular cross-section, e.g., an equilateral triangular cross-section when viewed in cross section taken along the length of the photobioreactor. Alternatively, one or more photobioreactors can have concave, hemispherical, trapezoidal or other shapes. Optionally, a single base panel and two or more covers can be joined (e.g., molded) together to form a single photobioreactor unit. For instance, a single base panel and three cover panels of triangular, hemispherical, trapezoidal or other shapes can be joined (molded, sealed, or otherwise coupled) to form a photobioreactor. Preferably, each photobioreactor is fabricated from materials (e.g., polymeric materials) adapted to resist leaching into the algae growth medium. In some embodiments, the polymer can be polyethylene terephthalate or polypropylene.

Certain embodiments of the invention also include a method of incubating algae growth medium through a photobioreactor system. The method can include the step of providing a photobioreactor system according to some embodiments. In a particularly preferred embodiment, for instance, the method can include the step of providing a photobioreactor system having pairs of photobioreactors, with each pair being used to support growth at a different phase of the algae growth cycle. The method can include providing a first sample of algae growth medium to an initial photobioreactor pair of the photobioreactor system. The first sample of algae growth medium can then be cycled either within the first pair of photobioreactors, or within a first photobioreactor itself, for a first interval of time. The first sample of algae growth medium can be moved from one or more upstream photobioreactors to one or more downstream photobioreactors. In turn, the first sample of algae growth medium can be cycled in or within one or more downstream photobioreactors for a second interval of time, e.g., in order to achieve a second growth phase. The sample of algae growth medium can, in turn, be moved from the downstream photobioreactors to one or more (e.g., a third pair of) photobioreactors further downstream. The sample of algae growth medium can be cycled in or within the third pair of photobioreactors for a third interval of time, e.g., in order to achieve a third growth phase. Finally, algal biomass can be harvested from the third pair of photobioreactors after the third interval of time.

Certain embodiments of the invention include a photobioreactor system for incubating algae growth medium, the system comprising one or more photobioreactors, each photobioreactor comprising: a substantially fluid-tight enclosure adapted to receive, incubate, agitate, and withdraw algae growth medium, the enclosure comprising one or more major surfaces that are substantially transparent to the transmission of electromagnetic radiation toward the algae growth medium, a plurality of aeration apertures positioned within each enclosure and adapted to deliver air bubbles in a manner sufficient to agitate the algae growth medium, one or more inlet and outlet ports associated with each photobioreactor, and the one or more photobioreactors each being supported and positionable in a manner sufficient to permit the gravity flow of algae growth medium from the outlet port of each photobioreactor, one or more light sources operatively and controllably associated with the photobioreactors to emit electromagnetic radiation at a direction of incidence toward the one or more major transparent surfaces, an aeration apparatus operatively and controllably connected to the plurality of aeration apertures in each photobioreactor.

### BRIEF DESCRIPTION OF DRAWINGS

The following drawings are illustrative of particular embodiments of the present invention and therefore do not limit the scope of the invention. The drawings are not necessarily to scale (unless so stated) and are intended for use in conjunction with the explanations in the following detailed description. Embodiments of the invention will hereinafter be described in conjunction with the appended drawings, wherein like numerals denote like elements.

FIG. 1 is a perspective view of an algal biomass production facility with a plurality of photobioreactor systems;

FIG. 2 is a perspective view of a photobioreactor system according to certain embodiments of the invention;

FIG. 3 is a front view of the photobioreactor system of FIG. 2;

FIG. 4 is a top view of the photobioreactor system of FIG. 2;

FIG. 5 is a side view of the photobioreactor system of FIG. 2;

FIG. 6 is a close-up side view of an upper portion of the photobioreactor system of FIG. 2;

FIG. 7 is a close-up perspective view of an upper portion of the photobioreactor system of FIG. 2;

FIG. 8A is a front view of a cover of a photobioreactor according to some embodiments of the invention;

FIG. 8B is a top view of the cover of FIG. 8A;

FIG. 8C is a side view of the cover of FIG. 8C;

FIG. 9A is a top view of a base panel of a photobioreactor according to some embodiments of the invention;

FIG. 9B is a front view of the base panel of FIG. 9A;

FIG. 9C is a side view of the base panel of FIG. 9A;

FIG. 9D is a perspective view of the base panel and the cover according to another embodiment;

FIG. 10A is a close-up perspective view of a base panel with aeration apertures according to some embodiments of the invention;

FIG. 10B is a cross-sectional view of the cross-section taken along C-C shown in FIG. 7;

FIG. 11 is a perspective view of a photobioreactor system according to some embodiments of the invention;

FIG. 12 is a schematic of two interconnected photobioreactors according to some embodiments of the invention;

FIG. 13 is a close-up view of a photobioreactor showing bubble formation in the photobioreactor according to some embodiments of the invention;

FIGS. 14A and 14B are various embodiments of a scraper system;

FIG. 15A-E is a schematic illustrating a method of circulating algae growth medium through a plurality of photobioreactors according to some embodiments of the invention;

FIG. 16A-F is a schematic illustrating a method of circulating algae growth medium through a plurality of photobioreactors according to some embodiments of the invention;

FIG. 17A-F is a schematic illustrating a method of circulating algae growth medium through a plurality of photobioreactors according to some embodiments of the invention;

FIG. 18 is a schematic illustrating a method of circulating algae growth medium through a plurality of photobioreactors according to some embodiments of the invention; and

FIG. 19 is a schematic illustrating a method of circulating algae growth medium through a plurality of photobioreactors according to some embodiments of the invention.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Examples of constructions, materials, dimensions, and manufacturing processes are provided for selected elements, and all other elements employ that which is known to those of ordinary skill in the field of the invention. Those skilled in the art will recognize that many of the noted examples have a variety of suitable alternatives.

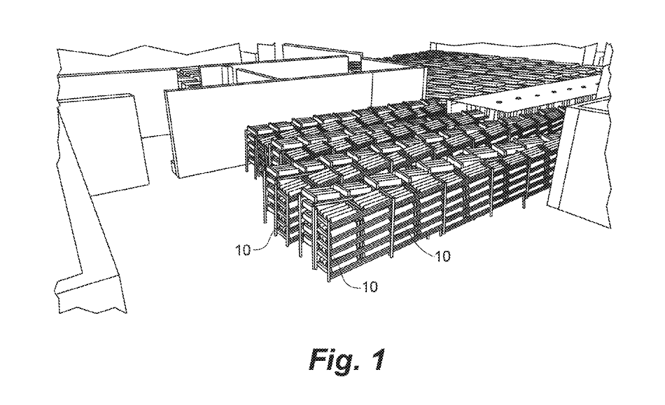

Certain embodiments of the invention include a photobioreactor system. The photobioreactor system can be useful for growing algae in any suitable location, e.g., within a building, in an enclosure, or on farms or open fields. The photobioreactor system can be operated continuously and/or cyclically to circulate algae growth medium through various components of the photobioreactor system. The growth medium can include water and nutrients to promote algal growth. For instance, with some species algal density can double once every 5 hours to about 30 hours. The pH of the growth medium can be monitored to ensure at least one sample of algae can be introduced in the water. Exemplary algae species that can be introduced into the water include Hp, Spirulina, and the like. The algae can be suspended in, and transported by, the recirculating growth medium through various stages of an algal production cycle. This can begin by introducing algae into the growth medium, and result in harvesting algal biomass from the photobioreactor system. As shown in FIG. 1, multiple photobioreactor systems can be used in a production facility for harvesting algal biomass. Each photobioreactor system 10 in such a production facility can operate independently of, or be interconnected with, one or more other photobioreactor systems.

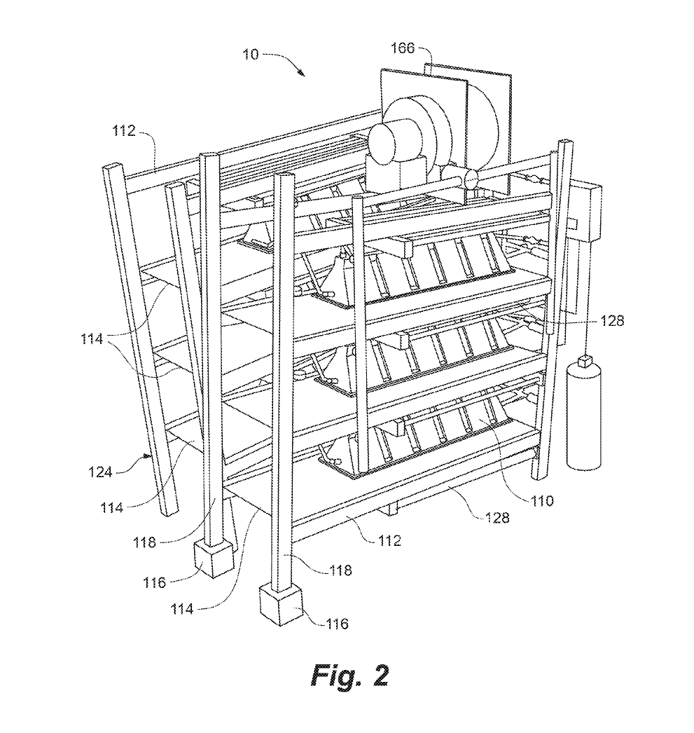

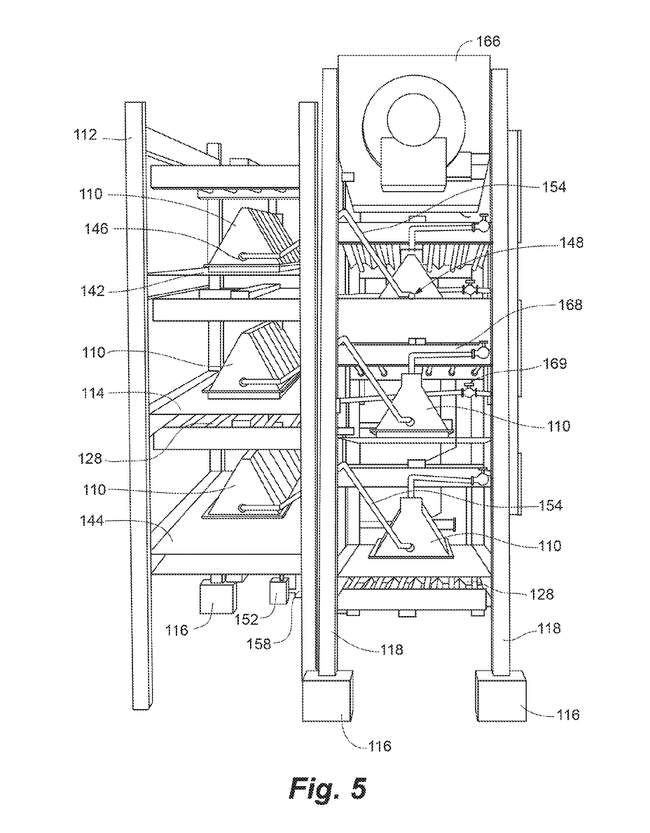

FIGS. 2 to 5 show various views of a photobioreactor system 10 according to some embodiments. As seen in FIG. 2, the photobioreactor system 10 includes a plurality of photobioreactors interconnected with each other. In the illustrated embodiment, the photobioreactors are positioned on a pair of shelving units 112. Alternatively, the photobioreactors can be suspended (e.g., from a ceiling) and supported by a frame and/or support surface. The photobioreactors can, alternatively, be placed on the ground. For instance, in one embodiment, the photobioreactors can be placed in series, and in a sloped fashion, thereby permitting the growth medium to flow at least in part by gravity, between and through one or more photobioreactor units. The photobioreactor system can therefore be used in an outdoor farm, or in an indoor production facility. The photobioreactor system can alternatively be used in a greenhouse.

Any number of photobioreactors can be used in the system in a modular fashion to scale up or scale down the algal biomass output. For instance, a photobioreactor system can have a single photobioreactor, e.g. for use in a home or educational setting. Additionally, or alternatively, the photobioreactor system can one or more pairs of photobioreactors. Each photobioreactor can be interconnected, and controlled independently of the other photobioreactors. Preferably, a photobioreactor system will have multiple pairs of photobioreactors, wherein each pair of photobioreactors includes a first photobioreactor interconnected with a second photobioreactor. In such systems, each pair can be operated independently. Alternatively, all pairs can be controlled and operated together by a central control system. A system of this invention can include any suitable number of photobioreactors (e.g., three, four, five, six, etc.) within a zone, all operably and independently coupled together in order to achieve desired growth characteristics based upon the requirements of the algal species. Each zone can be interconnected with another zone of photobioreactors and all zones of photobioreactors can be controlled together by a central control system. Alternatively, each zone can be controlled independently of each other. Such embodiments allow maximizing output from a farm, greenhouse or production facility by simply adding additional photobioreactor systems. Such embodiments can facilitate scaling the capacity of a facility based on demand or availability of resources such as light and water.

While not illustrated, one or more control systems can control one or more photobioreactors. The control system can measure, supply, monitor, and/or regulate a variety of parameters such as air, water, nutrients, pressure, temperature, pH, light, and the like. The control system can include measuring equipment for measuring a variety of parameters such as flow rate of air and water, pH, nutrients, algal density, light output, temperature, pressure and the like. The control system can also include a controller (e.g., a computer with a software program) for measuring, monitoring, storing, and regulating such parameters.

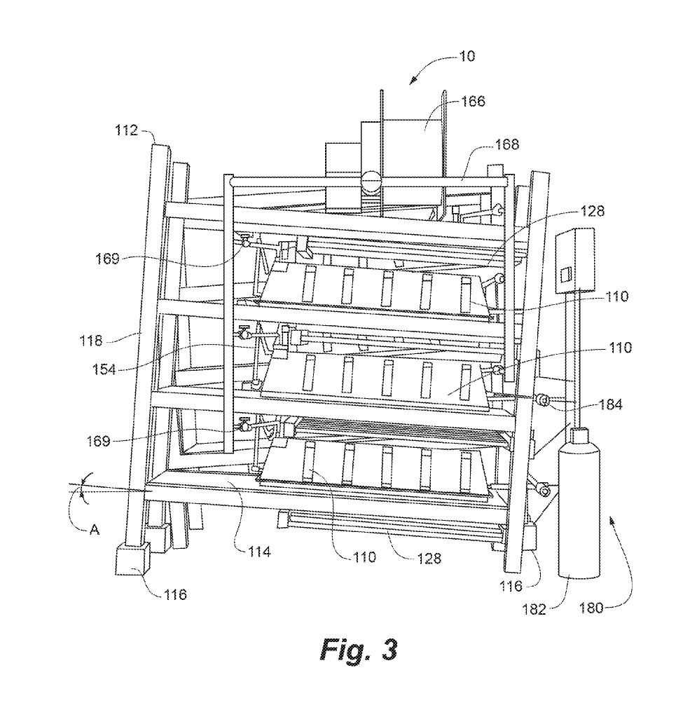

An algae growth medium can include algae inoculated into water and nutrients within a photobioreactor. Each shelving unit includes a number of shelves 114, with a photobioreactor 110 positioned in each shelf. The shelves 114 can be planar, but sloping such that the planar surface of each shelf forms an angle ‘A’ of about 1 degree to about 10 degrees with the horizontal plane (e.g., ground surface). As shown in the illustrated embodiment seen in FIGS. 2 and 3, the shelves 114 can be angled by positioning blocks 116 near the two legs 118 of the shelving unit, such that one side 120 of the shelving unit 112 is at a greater height than a second side 122 of the shelving unit 112. Alternatively, the shelves 114 can be angled with respect to the frame 124 of the shelving unit 112 and connected thereto by fasteners.

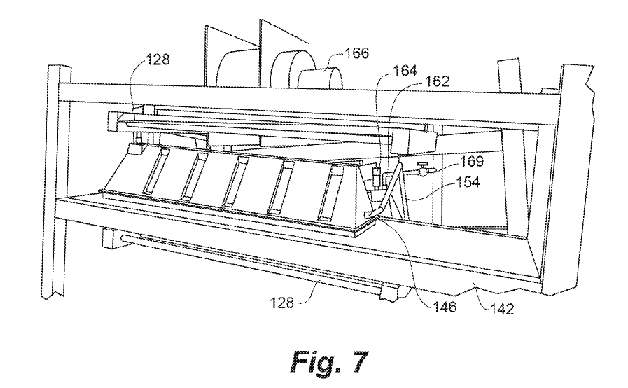

With continued reference to FIG. 2, and as best seen in FIG. 7, one or more light sources 128 in proximity to the photobioreactors supply electromagnetic radiation in the desired spectral range to promote algal growth. The photobioreactors have at least one major surface transparent to electromagnetic radiation to permit passage of radiation toward the algae growth medium. The light source 128 typically includes a plurality of light emitting diodes (LED) emitting radiation emulating sunlight in its spectral characteristics. For instance, the light source 128 can emit radiation in the spectral range corresponding to ultraviolet to infrared radiation. In alternate embodiments, the light source 128 can include a plurality of fluorescent lamps. Alternatively, a light source 128 emitting electromagnetic radiation in any spectral range can be used. As seen in FIG. 2, each shelf of the shelving unit includes one or more strip lights attached thereunder as the light source 128. The strip lights can produce 32 to 35 watts of electromagnetic radiation. Alternatively, any type of light source in a light fixture can be operatively engaged to the shelves 114 to provide electromagnetic radiation in the desired spectral range. Additionally, or alternatively, natural sources of electromagnetic radiation (e.g., ambient or concentrated sunlight) can be used.

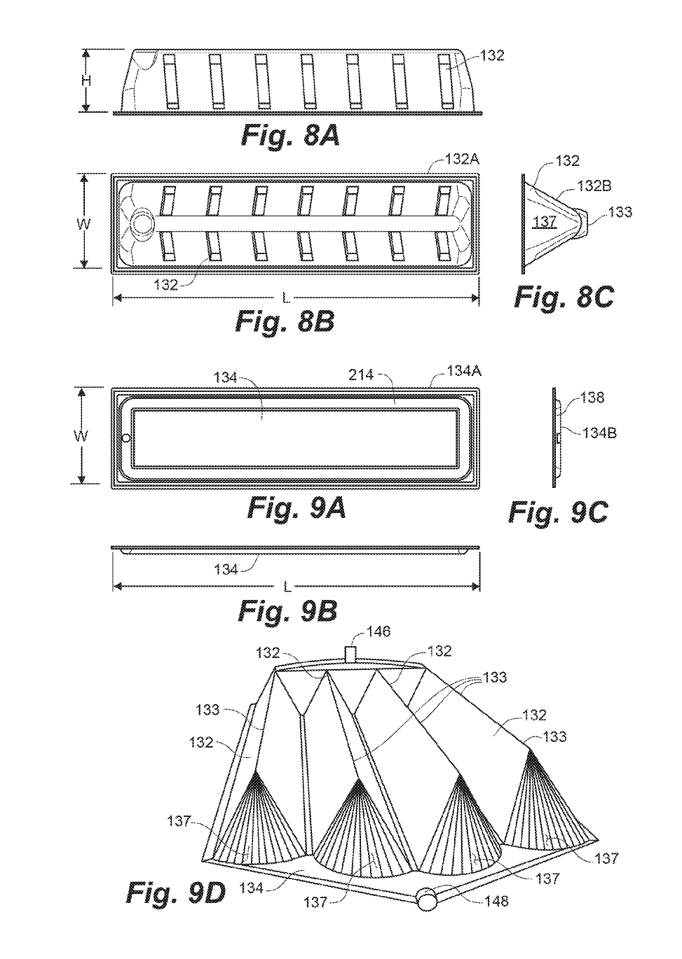

Referring now to FIGS. 8A-8C, and 9A-9C, the photobioreactor 110 comprises a cover 132 and a base panel 134. As seen in FIG. 8A-8C, the cover 132 and base panel 134 can be coupled to each other along at least a portion of their edge perimeters 132A, 134A. For instance, the cover 132 and the base panel 134 can be connected in the manner of a clamshell. Alternatively, or in addition, the cover 132 and the base panel 134 can be coupled in a hinged fashion such that the cover 132 can be lifted away from or moved toward the base panel 134. When connected, the cover 132 and the base panel 134 define a fluid tight enclosure. The surfaces of the cover 132 and the base panel 134 bound the enclosure. At least one major surface can be substantially transparent (e.g., transmittance of at least about 50%, and more preferably at least about 60%), such that the electromagnetic radiation can be directed toward the enclosure.

The cover 132 and the base panel 134 can be made of polymers such as polyethylene or polypropylene. Alternatively, the cover 132 and the base panel 134 can be made of polyvinylchloride. Sheets of polymer of thickness between about 1 millimeter and about 5 millimeters can be formed in any suitable manner, e.g., vacuum formed, injection molded, or blow molded into the cover 132 and the base panel 134 having desired shape and dimensions. In some embodiments, at least one major surface of the cover 132 or at least one major surface of the base panel 134 is transparent in the spectral range corresponding to ultraviolet, visible, and infrared electromagnetic radiation.

Referring back to FIGS. 8A-8C, and 9A-9C, in certain embodiments, photobioreactors are prism shaped. The cover 132 and the base panel 134 can each have respective edge perimeters (i.e., cover edge perimeter 132A and base edge perimeter 134A) and respective end perimeters (e.g., cover end perimeter 132B and base end perimeter 132B). In the illustrated embodiment, the edge perimeters 132A and 134A represent the perimeters of the outermost rectangular portions of the cover 132 and the base panel 134. The end perimeters 132B and 134B represent the perimeters of the lateral surfaces 137, 138 of the cover 132 and base panel 134.

In certain embodiments, the cover 132 and the base panel 134 form a triangular shape. Alternatively, the cover 132 and the base panel 134 can be of any suitable shape, such as pyramidal, concave, hemispherical, trapezoidal, or irregular shapes. The cover 132 and the base panel 134 can be sloped, curved, angled, bent or otherwise be oriented with respect to a horizontal plane in order to intercept electromagnetic radiation from a number of directions. Additionally, or alternatively, as shown in FIG. 9D, a plurality (e.g., two, three, four, or more) of covers 132 can be coupled to a single base panel 134. The cover 132 and the base panel 134 can form an equilateral triangular cross-section when viewed along the length of the photobioreactor. The equilateral triangular cross-section can be beneficial in offering algae optimal light exposure. However, the cover 132 and the base panel 134 can have any other shape (e.g., hemispherical, pyramidal, cuboidal etc.) without limiting the scope of the invention. The cover 132 can have a ridge 133. Referring back to FIGS. 8A-8C, 9A-9C, and the prism-shaped photobioreactors have a length “L”, width “W” and height “H”. In certain embodiments, the length L of the photobioreactor 110 is about 1 meter. In some embodiments, the width W of the photobioreactor 110 is about 30 centimeters. In some embodiments, the height H can be about 20 centimeters. Alternatively, the length, width and height can be in the range of about 1 meter to 2 meters, 25 centimeters to 1 meter, and 10 centimeters to 40 centimeters respectively.

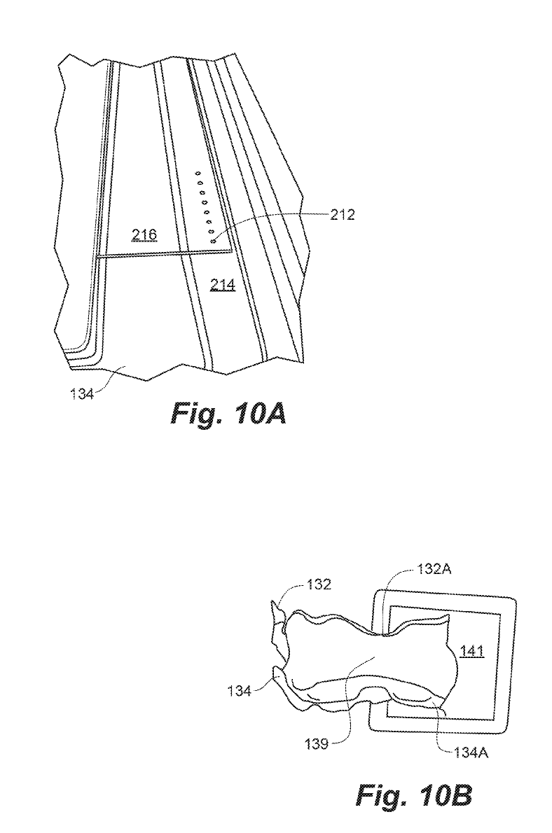

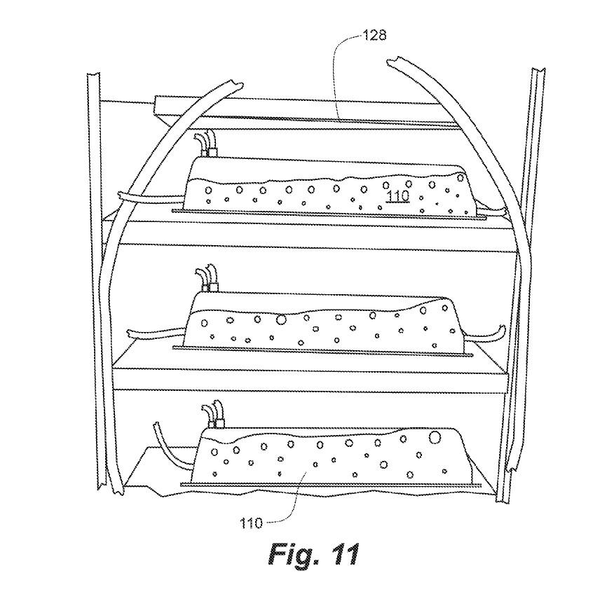

As seen in FIGS. 7 and 11, the cover 132 and the base panel 134 can be sealably connected together to form a substantially fluid-tight seal. In one example, the cover 132 and the base panel 134 can be bonded to form a substantially fluid-tight seal. For instance, the cover 132 and the base panel 134 can be bonded together by adhesives (e.g., a silicone adhesive). In such embodiments, an adhesive configured for forming a fluid tight seal between the cover 132 and the base panel 134 along their edge perimeters can be applied on the cover 132, the base panel 134 or both the cover 132 and the base panel 134. When bonded, the cover 132 and the base panel 134 form a fluid-tight enclosure for recirculating growth-medium in the photobioreactor system 10. Alternatively, the cover 132 and the base panel 134 can be bonded by a plastic weld. In alternative embodiments, the cover 132 and the base panel 134 can be attached instead of being bonded. For instance, the cover 132 and the base panel 134 can be attached by a plurality of fasteners (e.g., bolt and nut, hook and eye fasteners, clamps, compression fittings, complementary connectors, magnetic or mechanical latches etc.). Alternatively, the cover 132 and the base panel 134 can be interspersed with a sealant. The sealant can be gaskets, O-rings, fluid-resistant coatings, thread sealing tape, etc. so that water or any other liquids and gases do not leak via the contact surface 136 between the cover 132 and the base panel 134. In one example, the base panel 134 and the cover 132 can be covered by silicone adhesive to form a fluid-tight seal. In addition, as seen in FIG. 10B, a gasket 139 made of inert materials such as rubber or synthetic polymers can be pinched between the base panel 134 and the cover 132. For instance, a channel 141 can be formed enclosing the edge perimeters of the base panel 134 and the cover 132. The gasket 139 can be positioned in the channel 141 and pinched to provide a fluid tight seal.

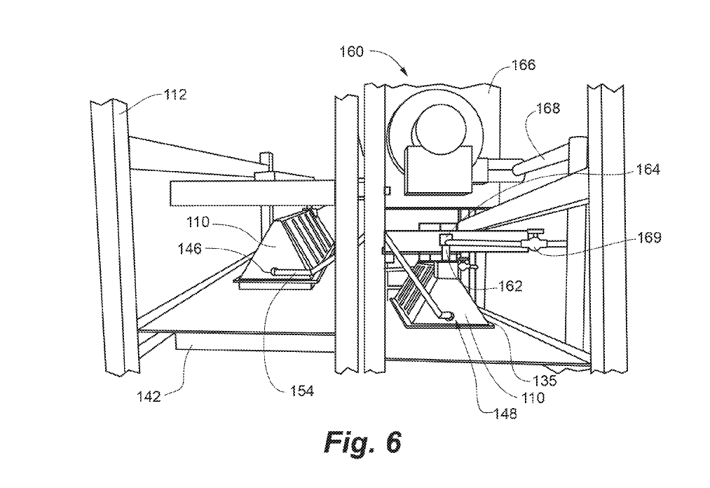

As described previously, the algae growth medium is circulated through the photobioreactors. A hydraulic circuit 140 can be used to facilitate supplying algae growth medium to one or more photobioreactors. Algae growth medium can flow by gravity from photobioreactors at the top shelves 142 of the shelving units 112 to the shelves 144. As shown in FIGS. 2 and 6, the cover 132 and the base panel 134 can have inlet and outlet ports 146, 148 defined therebetween. For instance, a top half of the inlet and outlet ports 146, 148 can be positioned on the cover 132, and a bottom half of the inlet and outlet ports 146, 148 can be positioned on the base panel 134. The inlet and outlet ports 146, 148 can be positioned on the side surface of the photobioreactor 110, as shown in FIGS. 2 and 6. Alternatively, the inlet and outlet ports 146, 148 can be positioned on any surface of the photobioreactor 110. In some embodiments, the stacking and interconnection of the photobioreactors facilitate gravity-induced recirculation of algae suspended in water. In such embodiments, the angled shelves facilitate fluid (e.g., water injected with algae) from moving downwardly from a photobioreactor 110 at the top shelf 142 to a photobioreactor 110 at the bottom shelf 144 due to gravity. Additionally, or alternatively, one or more water flow pumps 152 can supply algae growth medium from the photobioreactors at the bottom shelf 144 to those at the top shelf 142. In some embodiments, fluid can leave a photobioreactor 110 via the outlet port of each photobioreactor 110. The fluid can then be fed by gravity to the inlet port of an adjacent photobioreactor 110 positioned below. In some embodiments, this process can be repeated until fluid reaches the photobioreactor 110 positioned on the bottom shelf 144. Once the fluid reaches the photobioreactor 110 on the bottom shelf 144, it can flow via the outlet port 148 of the photobioreactor 110 on the bottom shelf 144 to the inlet port 146 of the photobioreactor 110 at the top shelf 142. In certain embodiments, the upwardly flow can be effected by a water flow water flow pump 152. Alternatively, an aquarium pump, a membrane pump, a peristaltic pump, or other pressure generating devices (e.g., pistons) can be used.

Those skilled in the art, given the present description, will appreciate the various ways in which a system as described herein can be used, based on the requirements and goals associated with their particular algae species. For instance, instead of a plurality of photobioreactors, a single photobioreactor may be provided. In such an embodiment, the photobioreactor may be slanted so as to permit gravity flow of the growth medium from the inlet port 146 of the photobioreactor to its outlet port 148. The water flow pump 152 may then pump the growth medium back into the photobioreactor. A photobioreactor system may include a plurality of such photobioreactors, each equipped with its own water flow pump 152 so that the growth medium circulates from the inlet port to the outlet port by gravity, and is then pumped back into the inlet port by each respective water flow pump.

As best seen in FIG. 7, and referring further to FIG. 11, the photobioreactors can be interconnected with each other to facilitate recirculation of algae growth medium. Each photobioreactor 110 can be connected to an adjacent photobioreactor 110 via a connecting tube 154. The connecting tube 154 can be polymer or thermoplastic tubes, hoses, pipes and the like. The connecting tubes 154 can be connected to the inlet and outlet ports 146, 148 on the cover 132 and the base panel 134 by frictional fit between the ports 146, 148 and the connecting tubes 154. Alternatively, the connecting tubes 154 can be connected to the ports 146, 148 via fluid fittings and adapters (e.g., threaded flow adapters, compression fittings, etc.). Additionally, the contact area between the inlet or outlet ports 146, 148 and the connecting tubes 154 can be sealed with sealing tape (e.g., polytetrafluoroethylene film, adhesives and the like). Alternatively, tube fittings such as barbed fittings forming a tight fit between the inlet or outlet ports 146, 148 and the connecting tubes 154. A clamp can then be placed on the connection. Each connecting tube 154 can be looped, and a fastener 156 can be placed at the top of the loop. One or more valves 158 can be in fluid communication with the connecting tubes 154. In certain embodiments, the valves 158 can be unidirectional. Alternatively, the valves 158 can be bidirectional. The valves 158 can be closed to hold algae growth medium in a given photobioreactor 110. The valves 158 can be opened to move algae growth medium from a given photobioreactor 110 (e.g., the photobioreactor 110 at the top shelf 142) to a different photobioreactor 110 (e.g., the photobioreactor 110 at the bottom shelf 144).

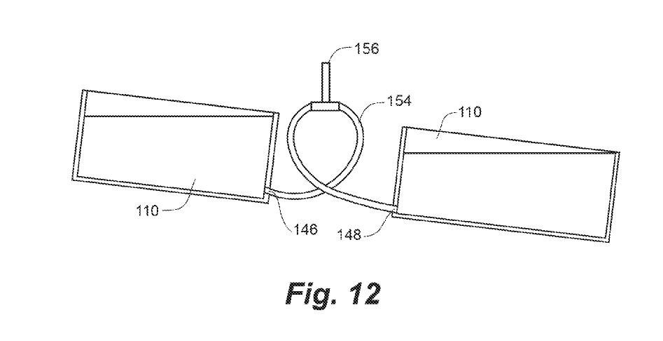

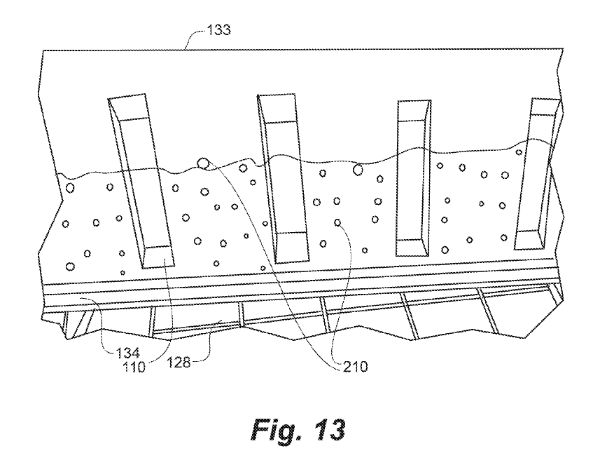

In certain embodiments, the connecting tube 154 can have a greater flow capacity than the water flow pumps 152 to prevent accumulation of water and/or overflow of water in each photobioreactor. As seen in FIG. 12, each connecting tube 154 is looped so that the top of the loop is positioned to correspond to the level of algae growth medium in the photobioreactors 110 at the top shelf 142. In certain embodiments, the loop is positioned at a maximum level of water in the photobioreactors 110 at the top shelf 142. In some embodiments, air at atmospheric pressure is introduced in the connecting tubes. As shown in FIG. 12, a fluid line can be positioned at the top of the loop to introduce air at atmospheric pressure. In the illustrated embodiment, the loop and the fluid line introducing atmospheric air form a “T” shaped joint 156. The fluid line introducing atmospheric air can be positioned above the level of fluid in the top photobioreactor 110 to ensure that the fluid line maintains a pressure equal to atmospheric pressure. The introduction of atmospheric air can eliminate gravity-induced motion of algae growth medium from a photobioreactor 110 at the top to one below (e.g., the connecting tube acting as a siphon). In some embodiments, if the level of liquid in the upstream photobioreactor 110 drops below the level of the loop, the flow between the photobioreactors can stop. At this point, further flow of fluid from the photobioreactor 110 on top can be induced by moving the loop to below the fluid level in the top photobioreactor 110. Such embodiments can allow the photobioreactor 110 on top to drain completely, thereby facilitating cleaning or replacing the photobioreactor 110.

In certain embodiments, best seen in FIGS. 3 and 6, an air circulation system 160 can introduce air into the photobioreactor 110 via air ports 162, 164 present on one or more surfaces on the first or bases 132, 134. Algae can absorb carbon dioxide present in air and convert the absorbed carbon dioxide into oxygen. When algae are exposed to carbon dioxide, water, nutrients, and light, they can photosynthesize and grow in the photobioreactor 110. As shown in FIGS. 3 and 6, a blower 166 can supply air via the air inlet ports 162 at the top surface of the cover 132 of each photobioreactor 110 via a plurality of air flow lines 168. One or more valves 169 can be in fluid communication with the air flow lines 168 supplying air to the photobioreactors. Once algae absorb and convert carbon dioxide into oxygen, air and/or oxygen can be vented to the atmosphere via air outlet ports 164. The blower 166 can generate sufficient pressure to push air through a plurality of photobioreactors. For instance, the blower 166 can generate a capacity of between about 0.01 cubic meters per second and about 0.1 cubic meters per second, preferably between about 0.02 cubic meters per second and about 0.03 cubic meters per second of air flow at a pressure of about 25 centimeters of water. Alternatively, or additionally, a vacuum system can be provided for venting air and/or oxygen from the photobioreactors. The vacuum system can draw air from the photobioreactors to the atmosphere via the air outlet ports 164. The resulting oxygen can be exhausted out of the air outlet port 164. Such embodiments can promote improved growth rates of photobioreactor 110, as oxygen residence in the photobioreactors can inhibit algae growth.

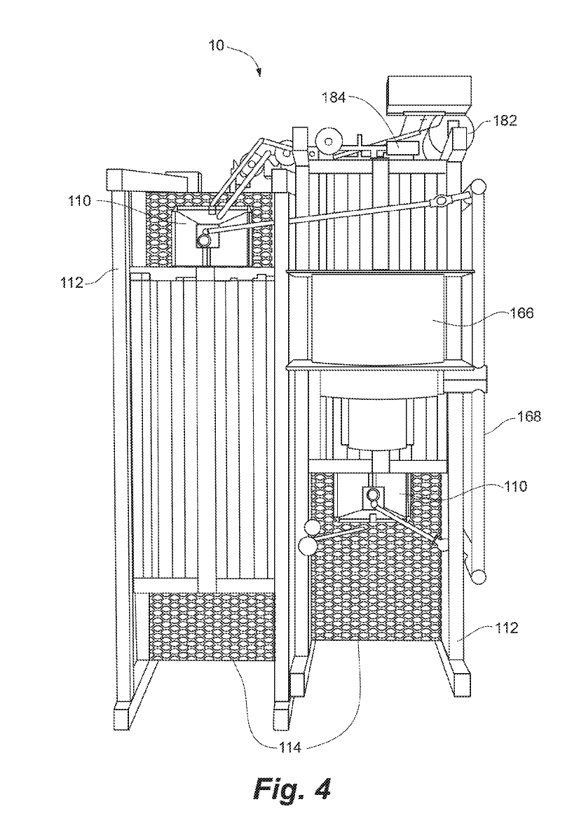

As algae grow in the photobioreactor 110, they can attach to the walls of the cover 132 and the base panel 134, thereby blocking light, air ports 162, 164, and/or inlet and outlet ports 146, 148. Certain embodiments of the invention can implement bubble aeration to prevent algae from occluding air ports, light and/or inlet and outlet ports. As shown in FIGS. 3 and 4, an aeration apparatus 200 can generate bubbles 210 of predetermined size via a plurality of aeration apertures 212 to keep algae suspended in the algae growth medium. The bubbles 210 cause an upward movement of algae thereby exposing algae to light from the light source 128 for at least a brief period of time. For instance, some micro-algae species such as Hp may need intermittent or non-continuous exposure to light for effective growth. The aeration apertures 212 can introduce turbulence in the growth medium, thereby preventing algae from growing, settling and/or depositing on any surface of the cover 132 or the base panel 134. The aeration apertures 212 can be designed with a diameter and spacing such that they agitate the growth medium and substantially prevent algae from clogging the aeration apertures 212. The aeration apertures 212 can agitate the growth medium such that algae from the interior of the enclosure defined by the base panel 134 and the cover 132 move outwardly toward the exterior. For instance, the algae can move toward a transparent major surface and be exposed to light from the light source 128 for a duration on the order of a few seconds (e.g., between about one second to about ten seconds). The aeration apertures 212 can continuously generate bubbles 210 so that algae from all areas of the enclosure are intermittently exposed to light.

The aeration apertures 212 can be situated on a channel near the top of the cover 132. In some embodiments, the aeration apertures 212 can be proximate the air inlet ports 162. Alternatively, as shown in FIG. 10, a channel 214 can be recessed in the base panel 134, and a cover 216 can be placed on the recessed channel 214. The aeration apertures 212 can be positioned on the cover 216. Alternatively, or additionally, a tube can supply air bubbles of desired size to each photobioreactor 110. The diameter and spacing of the aeration apertures 212 result in aeration (e.g., bubble formation) with desired bubble characteristics (e.g., bubble size and residence time in the photobioreactor 110). In some embodiments, the interconnection of photobioreactors can be configured to increase residence time of air bubbles 210 in the photobioreactors. In some embodiments, the bubbles 210 can substantially cover the surface area of the cover 132 of the photobioreactor 110. In some embodiments, the bubbles 210 can substantially cover the surface area of the cover 132 and the base panel 134 of the photobioreactor 110. The fluid lines supplying water and connected to the inlet and outlet ports 146, 148 of the photobioreactors can be made long and tortuous so as to allow air-bubbles 210 to stay for at least several seconds in each photobioreactor 110. Such embodiments allow optimal absorption of carbon dioxide by the algae. In some embodiments, surface area of the bubbles 210 can be tuned so as to maximize absorption of carbon dioxide.

In an exemplary embodiment, the diameter of the bubbles 210 can be about 0.01 centimeters. Alternatively, the diameter of the bubbles 210 can be between about 0.01 centimeters and about 0.05 centimeters. Alternatively, the diameter of the bubbles 210 can be between about 0.01 centimeters and about 0.1 centimeters. Alternatively, the diameter of each bubble 210 can be variable, and vary between about 0.01 centimeters and about 0.1 centimeters. The bubbles 210 can grow or shrink while in the enclosure. Each of the plurality of bubbles 210 can be of equal diameter. Alternatively, each of the plurality of bubbles can have a diameter different from the diameter of the other bubbles.

The end-to-end spacing between adjacent aeration apertures of the plurality of aeration apertures can be about 0.01 centimeters to about 1 centimeter, and preferably 0.05 centimeters to about 0.1 centimeter. A typical photobioreactor of the type described herein (e.g., on the order of 0.5 meters to about 10 meters in length, or preferably about 1 meter to 2 meters in length) can have between about 100 and about 1000, more preferably between about 200 and about 800, and even more preferably between about 300 and about 600 aeration apertures per photobioreactor of the type described herein. The spacing between adjacent aeration apertures may be constant or variable for all the aeration apertures.

Referring back to FIGS. 2-5, in some embodiments, a carbon dioxide supply system 180 can be operatively coupled to the aeration apparatus 200 to supply carbon dioxide into the growth medium. For instance, some species of algae may need an acidic growth medium (i.e., pH less than 7.0) for growth and photosynthesis. In such cases, algae can release oxygen back into the growth medium upon photosynthesis, thereby making the growth medium more basic (i.e., pH greater than 7.0). As a result, further growth and photosynthesis can be inhibited, and to prevent the growth medium from having a pH greater than about 7.0, carbon dioxide from a pressurized tank 182 can be supplied to the growth medium and result in further aeration. In some cases, a pressure regulator 184 can reduce the pressure of carbon dioxide from the pressurized tank prior to supplying it to the growth medium.

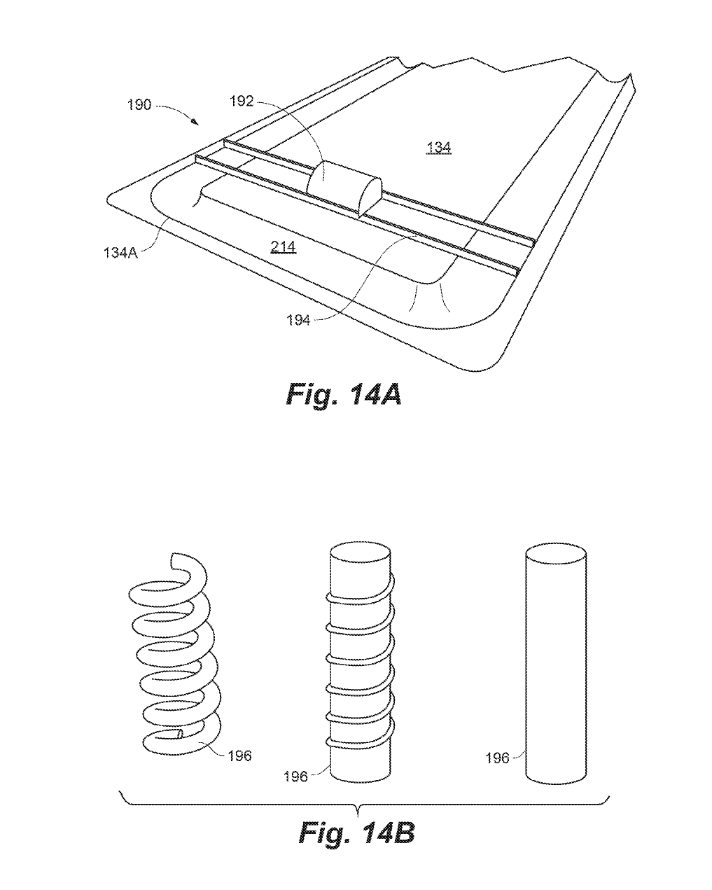

In alternate embodiments, one or more scrapers can be provided to scrape algae deposited on the bottom of the photobioreactor 110. Certain species of algae can have a tendency to self-flocculate and settle at the bottom of the photobioreactor 110. Additionally algae can settle on the substantially transparent major surface (e.g., bottom surface of the base panel 134) and block electromagnetic radiation from passing through the major surface. In such cases, a scraper can scrape algae settled in the bottom of the photobioreactor 110 (e.g., on the base panel 134) to prevent contamination and allow passage of electromagnetic radiation through the major surface. FIGS. 14A and 14B illustrate various embodiments of a scraper system 190. In the embodiment illustrated in FIG. 14A, a magnetically manipulated scraper 192 can travel width-wise (e.g., along direction “a”) in tracks 194. The tracks 194 can be positioned at various positions along the length of the base panel 134. For instance, after the scraper 192 has scraped algae settling at a first location, the scraper 192 and the tracks 194 can be moved (e.g., manually by an operator) to a different location along the length of the base panel 134. The scraper 192 can then be magnetically actuated to scrape algae settling at a different location. In other embodiments (not shown) the scraper 192 can be electrically or mechanically actuated.

FIG. 14B illustrates a scraper 196 according to a second embodiment. The scraper 196 is cylindrical. In other embodiments, the scraper 196 can be spherical. The scraper 196 can be neutrally buoyant, and can float or suspend in the growth medium in each photobioreactor. The scraper 196 can move upwards toward the top of the photobioreactor due buoyancy, turbulence in the agitated growth medium or due to bubble movement. As the scraper 196 moves in the photobioreactor 110 between the bottom surface where algae can settle, to the top surface, the motion results in scraping of the settled algae.

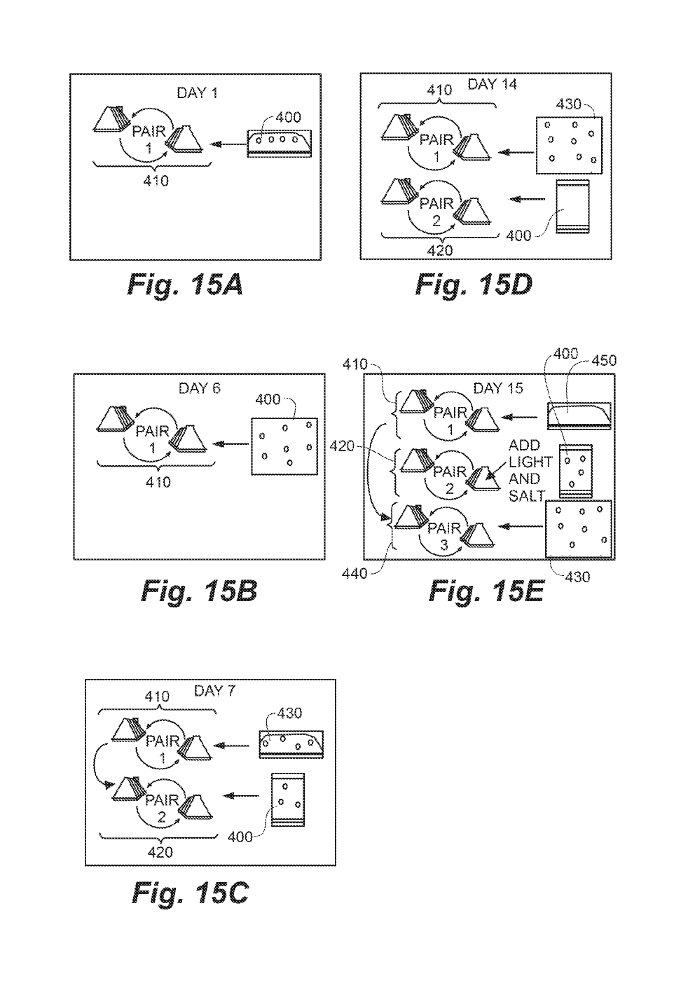

Embodiments of the invention also include a method of moving algae growth medium through the photobioreactors. In certain embodiments, algae can be introduced at the photobioreactors 110 at the top shelf 142 and algal biomass can be harvested from the middle or bottom-most photobioreactors. Algae growth medium can be circulated through the photobioreactors in the shelving units 112 shown in FIGS. 2 and 3, in a plurality of ways. As the algae move through the photobioreactors, the density of algae increases in each photobioreactor 110. An exemplary method of circulating algae through the photobioreactors is shown in FIGS. 15-17 and explained further with reference to the flowchart 300 shown in FIG. 18. A first sample of algae growth medium 400 (e.g., algae injected into water comprising nutrients, or simply algae and water) can be introduced at the photobioreactors 110 at the top shelf 142 at the beginning of the cycle (e.g., on day 1). Algae growth medium can cycle between the photobioreactor 110 at the top shelf 142 and an adjacent second photobioreactor 110 positioned immediately below the photobioreactors 110 at the top shelf 142 referred to herein as the first pair of photobioreactors 410. Algae growth medium can move downwardly toward the second photobioreactor 110 due to gravity, and upwardly toward the first photobioreactor 110 induced by a water flow water flow pump 152. As algae growth medium continues to cycle between the first and second photobioreactors, algae photosynthesize and grow. The density of algae increases in the first and second photobioreactors. The increasing density can result in a color change (e.g., green) in the first and second photobioreactors. In the illustrated embodiment shown in FIGS. 15A-15B, algae growth medium can cycle between the first and second photobioreactors for about six days.

Once algae reach the desired density, the first sample of algae growth medium 400 can be transferred from the first pair of photobioreactors 410 to the second pair of photobioreactors 420, as shown in FIG. 15C. The second pair of photobioreactors 420 can be positioned below the first pair of photobioreactors 410. On the day algae growth medium is moved from the first pair of photobioreactors 410 to the second pair of photobioreactors 420, a second sample of algae growth medium 430 can be introduced in the first pair of photobioreactors 410. The second sample of algae growth medium 430 can cycle through the first pair of photobioreactors 410 as the first sample of algae growth medium 400 can cycle through the second pair of photobioreactors 420. The density of algae increases in the first and second pairs of photobioreactors 410, 420. As shown in FIG. 15D, the second sample of algae growth medium 430 can cycle in the first pair of photobioreactors 410 for about a week. The first sample of algae growth medium 400 can cycle in the second pair of photobioreactors 420 during that time.

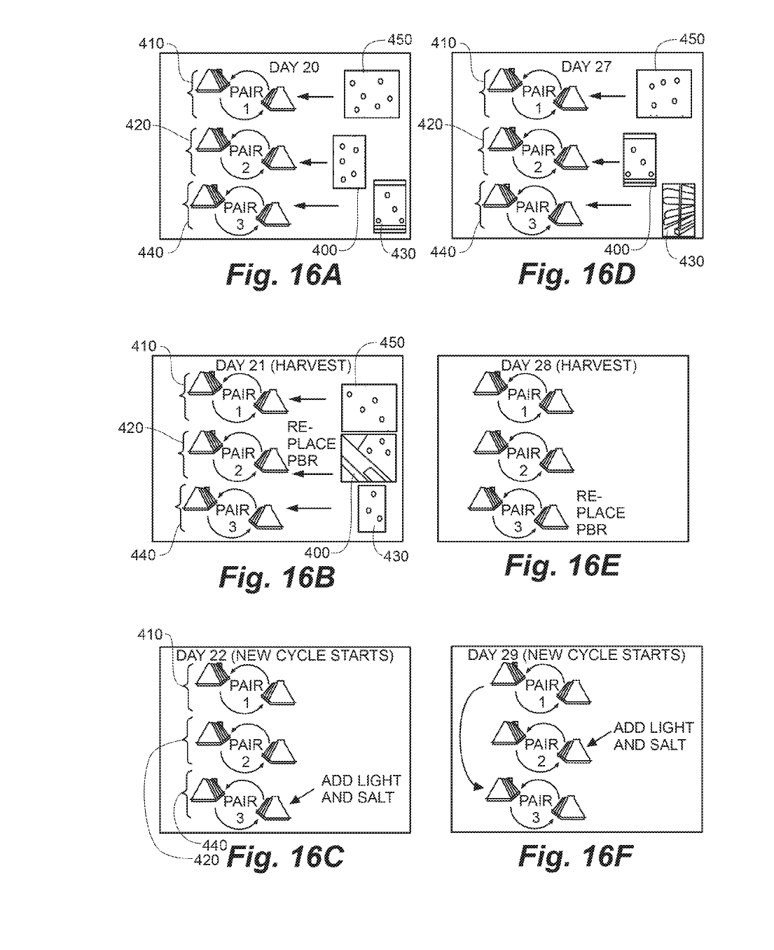

As shown in FIG. 15E, after the second sample of algae growth medium 430 has cycled in the first pair of photobioreactors 410 for about seven days, the second sample of algae growth medium 430 and water can be transferred to a third pair of photobioreactors 440. The third pair of photobioreactors 440 can be positioned below the second pair of photobioreactors 420. Once the second sample of algae growth medium 430 is removed from the first pair of photobioreactors 410, water injected with a third sample of algae growth medium 450 can be introduced into the first pair of photobioreactors 410. As shown in FIG. 16A, the first pair of algae growth medium 410 can continue to grow in density in the second pair of photobioreactors 420 in the meantime. As they grow, the second pair of photobioreactors 420 can have a color change from pale to dark green, and further from dark green to brown, as algae become “stressed” in the second pair of photobioreactors 420. The first sample of algae growth medium 400 can cycle through the second pair of photobioreactors 420 for about two weeks, before generating sufficient algal biomass to be harvested.

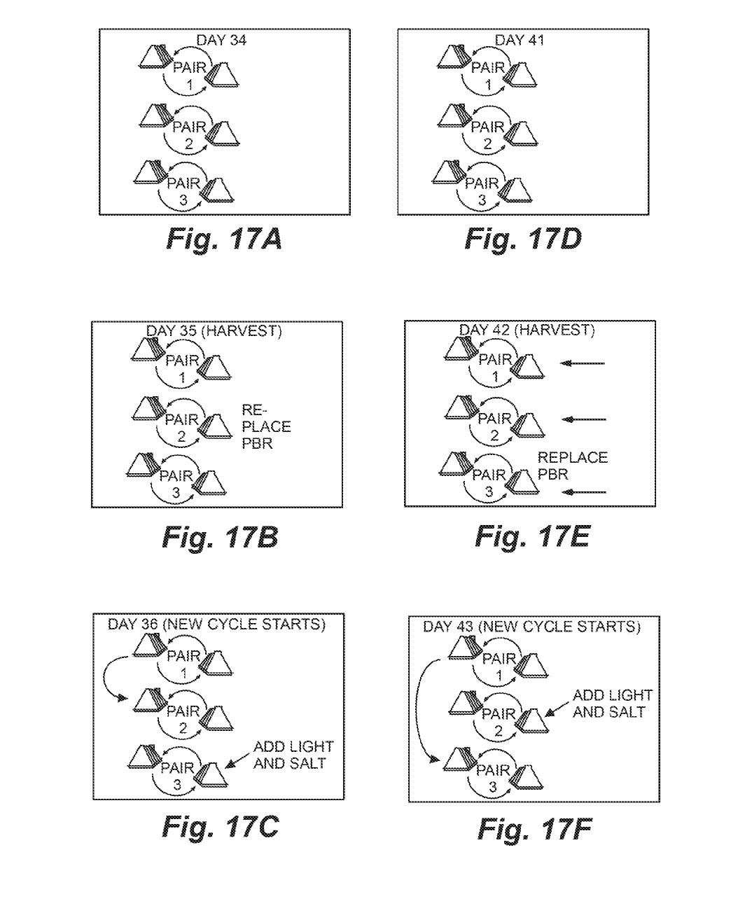

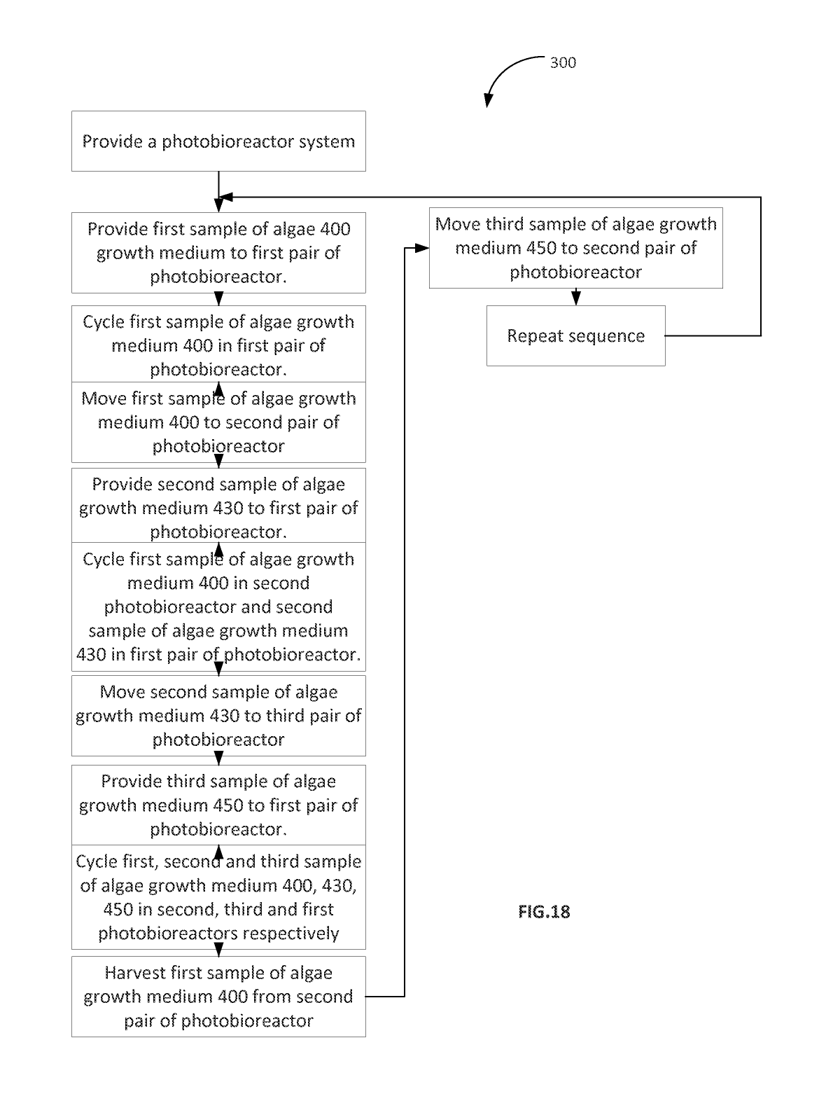

As shown in FIG. 16B, the first sample of algae growth medium 400 is harvested, and the third sample of algae growth medium 450 can be transferred to the second pair of photobioreactors 420. Alternatively, or additionally, the second pair of photobioreactors 420 can be cleaned and/or replaced prior to introducing the third sample of algae growth medium 450. After about a week, the second sample of algae growth medium 430 cycling through the third pair of photobioreactors 450 can become stressed as shown in FIGS. 16C-16D, and generate sufficient quantity of algal biomass so that they can be harvested as shown in FIGS. 15E. The process shown in FIGS. 15E, 16A-16D can then be repeated, as shown in FIGS. 16F, 17A-17F, thereby facilitating operation of the photobioreactor system to generate a target weight of algal biomass. It must be noted that the residence time of algae in photobioreactors in the above description can be increased or decreased by about one day to about four days.

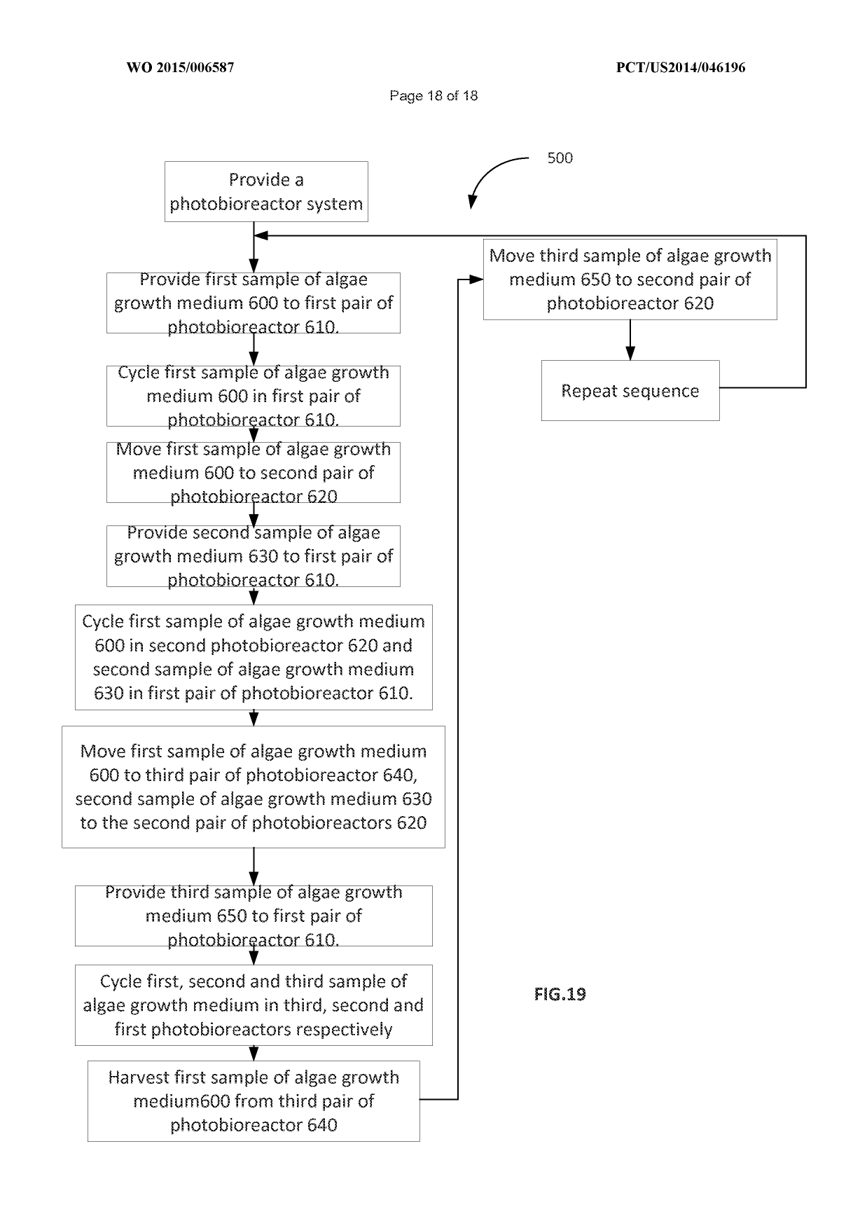

Alternatively, an exemplary method of moving algae growth medium through the photobioreactors can involve the steps shown in the flowchart 500 of FIG. 19, and as seen in FIGS. 2 and 3. A first sample of algae growth medium 600 can be introduced together with water at the first pair of photobioreactors 610. The first sample of algae growth medium 600 can cycle through the first pair of photobioreactors 610 for about a week, and continue to grow in density in the first pair of photobioreactors 610. As a result, the first pair of photobioreactors 610 exhibits a color change from clear transparency to pale green. At the end of a week, the first sample of algae growth medium 600 can be moved to the second pair of photobioreactors 620 placed below the first pair of photobioreactors 610.

Once the first sample of algae growth medium 600 is moved to the second pair of photobioreactors 620, a second sample of algae growth medium 630 and water can be introduced in the first pair of photobioreactors 610. The first sample of algae growth medium 600 cycles through the second pair of photobioreactors 620 for about a week before being transferred through to the third pair of photobioreactors 640 located below. Once the first sample of algae growth medium 600 is transferred to the third pair of photobioreactors 640, the second sample of algae growth medium 630 can be transferred to the second pair of photobioreactors 620, and a third sample of algae growth medium 650 can be introduced to the first pair of photobioreactors 610. The first sample of algae growth medium 600 can reside in the third pair of photobioreactors 640 for about a week before being harvested. The process can then be repeated to continuously produce algal biomass. It must be noted that the residence time of algae in photobioreactors in the above description can be increased or decreased by about one day to about four days.

While not illustrated, other methods of circulating the growth medium can involve operating each photobioreactor independently. In other words, algae can be introduced in each photobioreactor in a plurality of photobioreactor, and allowed to photosynthesize and grow until they reach a sufficient density. Once the target density is reached, algae and/or algal biomass can be harvested from each photobioreactor. In this example, each photobioreactor can be sloped, so that the growth medium enters the photobioreactor via the inlet port a first photobioreactor, and flow toward the outlet port of the first photobioreactor due to gravity. The growth medium can then be pumped from the outlet port back into the inlet port of the first photobioreactor. This process can be repeated until the algae density has reached a target density and harvested thereafter.

In another embodiment (not illustrated), the growth medium can flow sequentially to a first photobioreactor to an n^{th} photobioreactor in a zone or a photobioreactor system, where ‘n’ represents the number of photobioreactors. In some embodiments, ‘n’ can equal six, eight, or ten photobioreactors. Each photobioreactors is sloped and positioned such that growth medium flows from a first photobioreactor to a second photobioreactor positioned below the first photobioreactor, from the second photobioreactor to a third photobioreactor positioned below the second photobioreactor until the growth medium reaches the photobioreactor positioned at a bottom most level. At this stage, the growth medium can be pumped back to the first photobioreactor by a water flow pump. The process can continue until a sufficient density of algae is reached after which algae and/or algal biomass can be harvested from each photobioreactor.

The growth medium can flow through the photobioreactors in an intermittent or a steady fashion. In the intermittent operation, growth medium is supplied via inlet port of a first photobioreactor of a plurality of photobioreactors. The growth medium circulates in one or more photobioreactors until harvest, after which new growth medium can be introduced. Alternatively, growth medium can continuously flow through the inlet port of the first photobioreactor of a plurality of photobioreactors, and a quantity of algae of sufficient density and/or algal biomass can be continuously harvested from one or more photobioreactors.

Embodiments of the invention can be useful to generate algal biomass, which is used in a variety of products, such as food, dietary supplement, fuel (e.g., biofuel), and livestock feed. As mentioned previously, biofuels based on algal biomass can result in more efficient land use than corn or soy based biofuels. As algae production relies on carbon dioxide absorption, when coupled with carbon capture methods, can result in a nearly zero net-emission fuel source. Additionally, algal biomass generated according to embodiments of the invention can be useful in aquaculture wherein waste from marine ecosystem is absorbed by algae for nutrients, and algal biomass is in turn fed to fish, thereby improving water quality. A system of the present invention can be used to grow algae, including micro-algae, of any suitable species or source.

Those skilled in the art, given the present description, will be able to determine an optimal combination of structural and functional characteristics within the scope of this invention, in order to grow particular algae species in the manner described herein. Such species will typically vary, for instance, in terms of growth characteristics over the entire growth cycle, including varying phase requirements (e.g., in terms of settling, cell fragility, and CO2 requirements), as well as growth requirements (e.g., pH, nutrients, light), and the ability to be harvested. In turn, those skilled with be able to determine appropriate production conditions, including CO2-consumption and an overall balance, water consumption, energy demands and consumption profiles, biomass productivity data, and nutrient balances.

The term “algae,” as used herein, is any autotrophic organism capable of photosynthesis that lives in water (either freshwater and/or seawater). The term “algae” includes “macroalgae” (seaweed) and “microalgae” (small algae), and can include diatoms (Bacillariophyceae), green algae (Chlorophyceae), blue-green algae (Cyanophyceae), golden algae (Chrysophyceae or chrysophyte), brown algae, and/or red algae. The algae can be any algae species including macro algae, micro algae, marine algae, or freshwater algae. Nonlimiting examples of suitable algae include chiarella vulgaris, haematococcus, stichochoccus, bacillariophyta (golden algae), cyanophyceae (blue green algae), chlorophytes (green algae), chlorella, botryococcus braunii, cyanobacteria, prymnesiophytes, coccolithophorads, neochloris oleoabundans, scenedesmus dimorphus, atelopus dimorphus, euglena gracilis, dunalielia, dunaliella salina, dunaliella tertiolecta, diatoms, bacillariophyta, chlorophyceae, phaeodactylum tricornutunum, stigmatophytes, dictyochophytes, and pelagophytes. The algae may be single cells, colonies, clumps, and any combination thereof. The algae can be natural algae, or can be genetically-modified algae, and the algae suspension may contain a monoculture (single algae species) or a multiculture (multiple algae species). In an embodiment, the algae suspension contains a monoculture. Some preferred embodiments of the invention are suitable for growing Dunaliella salina, Chlorella vulgaris/emersonii. Spirulina platensis, Haematococcus pluvialis, Odontella sp., Porphyridium cruentum, Shizochytrium sp., Isochrysis galbana, Scenedesmus obliquus, Nannochloropsis sp.

Thus, embodiments of the invention are disclosed. Although the present invention has been described in considerable detail with reference to certain disclosed embodiments, the disclosed embodiments are presented for purposes of illustration and not limitation and other embodiments of the invention are possible. One skilled in the art will appreciate that various changes, adaptations, and modifications can be made without departing from the spirit of the invention.

1. A photobioreactor system for incubating algae growth medium, the system comprising:
one or more photobioreactors, each photobioreactor comprising:
a substantially fluid-tight enclosure adapted to receive, incubate, agitate, and withdraw algae growth medium,
the enclosure comprising one or more major surfaces that are substantially transparent to the transmission of electromagnetic radiation toward the algae growth medium,
a plurality of aeration apertures positioned within each enclosure and adapted to deliver air bubbles in a manner sufficient to agitate the algae growth medium,
one or more inlet and outlet ports associated with each photobioreactor, and
the one or more photobioreactors each being supported and positionable in a manner sufficient to permit the gravity flow of algae growth medium from the outlet port of each photobioreactor,
one or more light sources operatively and controllably associated with the photobioreactors to emit electromagnetic radiation at a direction of incidence toward the one or more major transparent surfaces,
an aeration apparatus operatively and controllably connected to the plurality of aeration apertures in each photobioreactor.

2. The photobioreactor system of claim 1, wherein the aeration apertures each, independently, have a diameter in the range of about 0.01 centimeters to about 0.05 centimeters.

3. The photobioreactor of claim 1, wherein the aeration apertures are each, independently, between about 0.1 centimeters and about 1 centimeter apart.

4. The photobioreactor of claim 3, wherein either the diameters of the apertures or the spacing between adjacent apertures, or both, are varied throughout the aeration apparatus.

5. The photobioreactor of claim 3, wherein either the diameters of the apertures or the spacing between adjacent apertures, or both, are identical throughout the aeration apparatus.

6. The photobioreactor of claim 1, wherein the air bubbles delivered by the aeration apertures agitate the algae growth medium in a manner sufficient to substantially prevent clogging of aeration apertures.

7. The photobioreactor of claim 1, wherein with the system in operation, the plurality of aeration apertures deliver air bubbles in a turbulent manner sufficient to prevent settling of algae at the bottom of each photobioreactor.

8. The photobioreactor system of claim 7, wherein the bubbles delivered by the aeration apertures have a residence time in the enclosure of between about one second to about two seconds.

9. The photobioreactor system of claim 1, wherein permits the flow of algae growth medium from a first photobioreactor to a second photobioreactor due to gravity.

10. The photobioreactor of claim 9, wherein each photobioreactor is sloped sufficiently to permit gravity flow of algae growth medium from the outlet port of each photobioreactor.

11. The photobioreactor system of claim 1, wherein each photobioreactor comprises
a base panel and a cover, the base panel and the cover each having an edge perimeter and an end perimeter, the base panel and the cover being sealably connected to each other at their respective edge perimeters to form a fluid tight seal, such that they define the substantially fluid tight enclosure.

12. The photobioreactor system of claim 11, wherein the bubbles generated by the aeration apertures move in an upward direction along one or more major surfaces from the base panel and toward a ridge of the cover.

13. The photobioreactor system of claim 11, wherein the aeration apertures are positioned in a channel recessed in the base panel.

14. The photobioreactor system of claim 11, wherein the base panel and the cover are sealably connected in a manner selected from the group consisting of physical connectors, adhesives, gaskets and combinations thereof.

15. The photobioreactor system of claim 14, wherein the base panel and the cover are sealably connected by the use of a silicone adhesive and a gasket interspersed between the base panel and the cover.

16. The photobioreactor system of claim 1, further comprising one or more water flow pumps each in fluid communication with a respective photobioreactor, the water flow pumps being adapted to transfer the growth medium from a downstream outlet port to an upstream inlet port of the same or different photobioreactor.

17. The photobioreactor system of claim 16, wherein the downstream outlet port and the upstream inlet port are associated with a single photobioreactor.

18. The photobioreactor of claim 16, wherein the downstream outlet port is associated with a first photobioreactor and the upstream inlet port is associated with a second photobioreactor.

19. The photobioreactor system of claim 1, wherein one or more photobioreactors are independently maintained at atmospheric pressure.

20. The photobioreactor system of claim 1, wherein the one or more major surfaces that are substantially transparent to electromagnetic radiation are sloped with respect to the direction of incidence of the electromagnetic radiation.

21. The photobioreactor system of claim 1, wherein each photobioreactor has a triangular cross-section.

22. The photobioreactor system of claim 1, wherein each photobioreactor is fabricated from materials comprising a polymer that is substantially resistant to leaching into the algae growth medium.

23. The photobioreactor system of claim 22, wherein the polymer is selected from the group consisting of polyethylene terephthalate and polypropylene.

24. The photobioreactor system of claim 1, wherein a plurality of photobioreactors are each associated with a corresponding algae growth phase.

25. The photobioreactor system of claim 1, wherein a plurality of photobioreactor pairs are each associated with a corresponding algae growth phase.

26. The photobioreactor system of claim 25, wherein a first pair of photobioreactors is associated with a first algae growth phase, a second pair of photobioreactors is associated with a second algae growth phase, and a third pair of photobioreactors is associated with a third algae growth phase.

27. A method of incubating algae growth medium, comprising the steps of:
providing a photobioreactor system according to any previous claim,
introducing the components of an algae growth medium into a first photobioreactor through its upstream inlet port,
incubating the algae growth medium within the first photobioreactor by a) operating the photobioreactor under predetermined conditions of time, temperature and pressure, b) directing electromagnetic radiation toward the algae growth medium through the one or more major surfaces, c) operating the aeration apparatus and plurality of aeration apertures in order to deliver air bubbles into and within the growth medium in a manner sufficient to agitate the algae growth medium while substantially preventing the clogging of aeration apertures, and
withdrawing the algae growth medium in a predetermined manner by the use of gravity flow from the exit port of the photobioreactor to the inlet port of the same or different photobioreactor.

28. A method of incubating algae growth medium through a photobioreactor system according to claim 26, comprising:
providing a first sample of algae growth medium to the first pair of photobioreactors;
cycling the first sample of algae growth medium in the first pair of photobioreactors for a first interval of time;
transferring the first sample of algae growth medium from the first pair of photobioreactors to the second pair of photobioreactors;
cycling the first sample of algae growth medium in the second pair of photobioreactors for a second interval of time; and
transferring the first sample of algae growth medium from the second pair of photobioreactors to the third pair of photobioreactors;
cycling the first sample of algae growth medium in the third pair of photobioreactors for a third interval of time; and
harvesting algal biomass from the third pair of photobioreactors after the third interval of time.

29. The method of claim 28, wherein the first, second, and third time intervals are each, independently, on the order of about one week.

30. The method of claim 29, wherein the first, second and third interval of time correspond to a duration when density of algae in the first, second and third pair of photobioreactors is about 0.1% by dry-weight of algal biomass.
